(19) United States
(12) Patent Application Publication
(10) Pub. No.: US 2011/0061653 A1
(43) Pub. Date: Mar. 17, 2011

(54) DISPENSER FOR PULVERULENT SUBSTANCES CONTAINED IN A SEPARATE PACK

(76) Inventor: Alfred Von Schuckmann, Kevelaer (DE)

(21) Appl. No.: 12/992,638

(22) PCT Filed: May 6, 2009
(86) PCT No.: PCT/EP09/55468

§ 371 Date: Nov. 15, 2010

(30) Foreign Application Priority Data
May 13, 2008 (DE) 10 2008 023 376.5

(51) Int. Cl. A61M 15/00

(52) U.S. Cl. 128/203.15; 128/203.21

(57) ABSTRACT

The invention relates to a dispenser for pulverulent compounds contained in a separate pack, in particular compounds contained in a blister pack, wherein the pack has a container part and a tear-off container cover, wherein, furthermore, an air flow aspirated after removal of the container cover from the container part is at least partially aspirated through the container part. The container cover projects out of a tear-off opening of the dispenser for actuating purposes. The dispenser of the type in question is improved in a structurally simple manner such that functionally reliable handling is provided over the course of the inhaling operation. The air flow is aspirated laterally into the dispenser with respect to a tear-off direction of the container cover, and that the container cover closes the tear-off opening.

The invention relates, in first instance, to a dispenser for pulverulent substances contained in a separate pack, in particular substances contained in a blister pack, the pack having a container part and a pull-off container covering, furthermore a sucked-in air stream being sucked, at least in part, through the container part, following removal of the container covering from the container part, and the container covering projecting out of a pull-off opening of the dispenser for actuating purposes.

Dispensers of the type in question are known. These serve predominantly for the inhalation of pulverulent medicament substances which are provided in portions in cavity-forming container parts in particular of a blister pack. The container parts filled with the substance which is to be inhaled are covered by a laminated-on aluminum foil. Also known in this respect, in particular, are portable dispensers in the form of inhalers, as known, for example, from U.S. Pat. No. 5,660,169. These inhalers contain a container part which is opened immediately prior to inhalation by virtue of the container covering being pulled off. This action of pulling off the container covering is facilitated by an end of the container covering which is exposed beyond a pull-off opening of the dispenser, so that, by virtue of this exposed end being subjected to pulling action, the container covering unrolls to uncover the container part. The inhalation process takes place by virtue of the air flow which is produced when the user breathes in during inhalation flowing through the container part, the stored medicament being drawn out of the container part and breathed in.

In respect of the above-described prior art, a technical problem of the invention is considered that of improving a dispenser of the type in question in a constructionally simple manner so as to achieve functionally reliable handling during the inhaling process.

This problem is solved first and foremost by the subject matter of claim 1, this being based on the fact that the air stream is sucked into the dispenser laterally, in relation to a pull-off direction of the container covering, and that the container covering closes the pull-off opening. This configuration gives a dispenser of the type in question which is distinguished by functionally reliable handling during the inhalation process. The air stream which draws the substance out of the container part is sucked in, during inhalation, on the far side of the container covering, which has been pulled off into the opening position for the container part, and, accordingly, it does not flow over this container covering, which, with corresponding air-stream action, can result in interference in the air flow for example as a result of the container covering flapping about. Flow over the container covering, furthermore, may result in the container covering standing up in an uncontrolled manner, which, ultimately, can even result in the air-inflow opening of the dispenser being closed by the container covering. The known arrangement may lead to the inhaling result being insufficient. This disadvantage is counteracted by the lateral inflow according to the invention. The air stream which clears out the container part, in this case, is guided specifically past the container covering and on to the container part, the container covering furthermore closing the pull-off opening and thus counteracting any intake of additional air. The closure of the pull-off opening by the container covering alone here is not necessarily air-tight. Rather, the sealing in practice is achieved, ultimately, by the negative pressure inside the device, this negative pressure being produced as the container part is cleared out and forcing the container covering with sealing action against the pull-off opening.

The invention also relates to a dispenser according to the features of the preamble of claim 1. In order to improve a dispenser of the type in question, it is proposed that the air stream, following passage through the container part, enters, in the same plane, into a deflecting portion of a vortex chamber, the vortex chamber extending over two levels and, furthermore, one or more additional outside-air openings opening out in the vortex chamber. As a result of this configuration, the pulverulent substance drawn out of the container part is distributed uniformly in the suction air which is to be inhaled. Configuring the vortex chamber over two levels gives rise to the vortex path being extended, and this, in addition, allows an arrangement in which the container part, the vortex chamber and the suction mouth are disposed one behind the other in the main throughflow direction. With a desired vortex-chamber extent of more than 180°, the vortex-chamber entrance may thus be disposed, at least in one projection, opposite the exit.

The features of independent claims 1 and 2 described above are pertinent both in themselves and in any combination, it also being possible to combine features of an independent claim 1 or 2 with the features of a further independent claim or with features of a number of independent claims, and also with just individual features of one or more of the further independent claims 1 to 2.

Further features of the invention will be explained hereinbelow, also in the description of the figures, often in their preferred association with the subject matter of claims 1 and/or 2 or with the features of further claims. However, they may also be important in association with just individual features of claim 1 and/or claim 2 or of the respective further claim or independently in each case.

Thus, in a development of the subject matter of the invention, it is provided that the incoming air passes, in first instance, through a collecting channel which splits up into a direct channel, which leads to a suction mouth, bypassing the container part in the process, and also into a container channel, which leads to the container part. The container channel terminates in a preferred configuration, prior to the container covering being pulled off, as a blind passage in the region of the container part, the latter, once the container covering has been pulled off, being part of the container channel, as a result of which the divided-off air flow guided via the container channel clears out the container part. It is preferable here for the divided-off air stream to penetrate through the container part. In a development of the subject matter of the invention, the air flows downstream of the collecting channel are divided up uniformly. However, non-uniform divisions are also possible, for example a division of 60% to 40% or 70% to 30% in respect of the distribution between the direct channel and the container channel.

In an advantageous configuration of the subject matter of the invention, it is provided that the container channel and the direct channel open out in the vortex chamber, so that, accordingly, the partial air streams combined here leave the suction mouth together. It is also preferred in this respect if the container channel and the direct channel combine upstream of the vortex chamber, so that the partial air streams enter into the vortex chamber in an already combined state. As a result of this configuration, the container channel is formed in the manner of a bypass channel in relation to the direct channel, further preferably upstream of the vortex chamber, as seen in the flow direction.

It is advantageously further provided that the container channel and/or the direct channel enter/enters into an upper level of the vortex chamber, which is formed over two levels. This results in flow taking place through the double-level vortex chamber from top to bottom. As an alternative, however, it is also possible for flow to take place into the lower level, and to exit from the upper level, of the vortex chamber.

An easy-to-handle configuration is achieved in that the dispenser is configured such that it can be swung open in order for the container part to be placed in position. The holder for the container part is thus easily accessible, and can thus also be exposed, in particular by virtue of the dispenser part being swung open, preferably over its entire surface area, this preferably also relating to the pull-off region of the container covering. The opening flap provided for this purpose can extend, furthermore, over the entire, or more or less entire, basic surface area of the dispenser. A preferred configuration in this respect is one in which the opening flap is formed on the pull-off side of the vortex chamber, i.e. merely on that side of the vortex chamber which is directed away from the suction mouth, and the dispenser part formed here has the holder for the container part and also has the pull-off opening and the air-entry opening. The vortex chamber preferably cannot be opened by the flap.

In a simple configuration, the opening flap may be a sheet-like component which is provided, in the manner of a lid, with a planar underside. A preferred configuration in this context, however, is one in which the opening flap has shaped structures which, in conjunction with the accommodating region for the container part, create the desired air paths. Accordingly, the air paths in the dispenser, in particular the air paths upstream of the vortex chamber, as seen in the flow direction, are formed by corresponding shaped structures of the opening flap and of the dispenser part which is fixed in relation to the flap. Furthermore, it is preferred if the opening flap is articulated on the dispenser such that it can be pivoted about a flap axis, which opening axis runs parallel to the pull-off direction of the container covering. In a development of the subject matter of the invention, the opening flap is transparent, and is further preferably made of a transparent plastics material, and in a preferred configuration the entire dispenser is a plastics injection molding.

The transparent configuration of the opening flap proves to be advantageous to the extent that, as a result thereof, a marking line is visible to the user, and this means that the container covering can be pulled off in an appropriate manner. The marking line here may be a constituent part of the dispenser. As an alternative, the marking line may also be part of the container part or part of the container covering. In any case, the marking line serves as a visual positioning aid for the user, and indicates to the user the minimum pull-off position of the container covering, in which minimum pull-off position the container part is reliably exposed over the entire opening surface area. This ensures that the container part is cleared properly and completely by the air flow guided through the container channel.

It is further preferable for a plurality of inlet openings, thus for example five to fifteen, preferably seven to ten, further preferably eight, inlet openings, to be formed for the incoming air, these openings, furthermore, being formed in particular in a groove-like manner. These inlet openings open out directly into the collecting channel.

Furthermore, the inlet openings are oriented preferably transversely to the direction in which the dispenser extends, in addition transversely to the pull-off direction of the container covering and thus also transversely to the longitudinal extent of the collecting channel, it also being the case that the inlet openings, covering over the collecting channel in a grid-like manner, open, in part, in the upward direction and, in part, laterally. In one configuration of the subject matter of the invention, the inlet'openings extend on the upper side over the entire width of the collecting channel, as seen transversely to the longitudinal extent of the collecting channel, whereas the inlet openings along the vertical extent of the collecting channel are formed only approximately over half the height of the same.

Finally, it is provided that the flow path of the lower level is formed, at least in part, inside the flow path of the upper level. Accordingly, in a plan view of the dispenser, the inner, lower flow path forms the eye of the upper flow path, further preferably in a configuration in which the air flow enters into the upper level and exits through the suction mouth via the lower level. This can result, as seen over two levels, in a helical flow within the vortex chamber. The central flow path of the lower level is preferably the partial flow path which is connected to the additional outside-air opening and is not laden with substance.

The numerical ranges given in each case also include—if not already given by way of example—all intermediate values, to be precise limited in increments of a tenth from the lower and/or upper limit in the direction of the respective other limit. “And” here means that both limits are shifted by one or more tenths in each case in the direction of the other limit, i.e. they are narrowed down.

The invention will be explained in more detail hereinbelow with reference to the accompanying drawing, which illustrates merely an exemplary embodiment and in which:

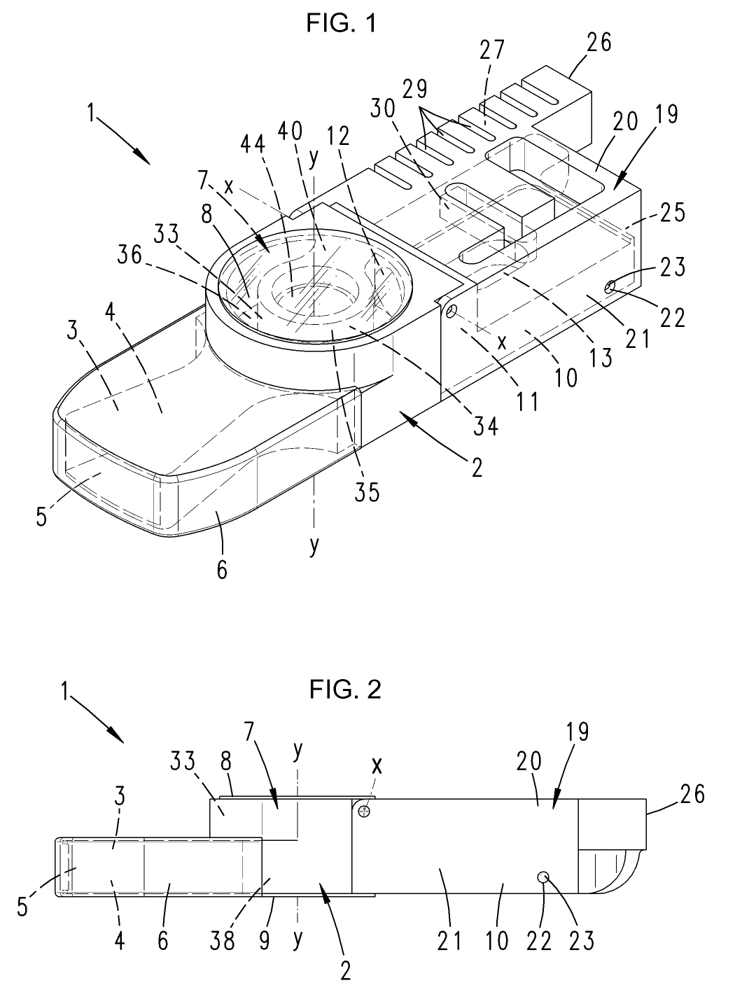

FIG. 1 shows a perspective illustration of a dispenser of the type in question in the not-in-use position;

FIG. 2 shows a side view of the dispenser;

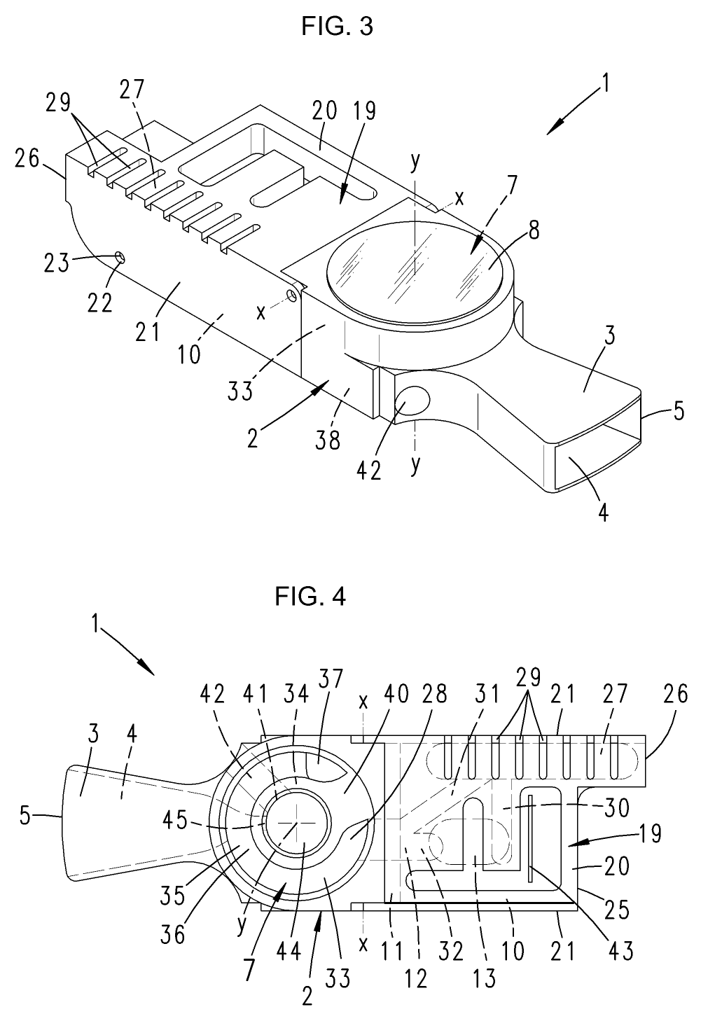

FIG. 3 shows a further perspective illustration of the dispenser, following removal of a protective cap for the suction mouth;

FIG. 4 shows the plan view in this respect;

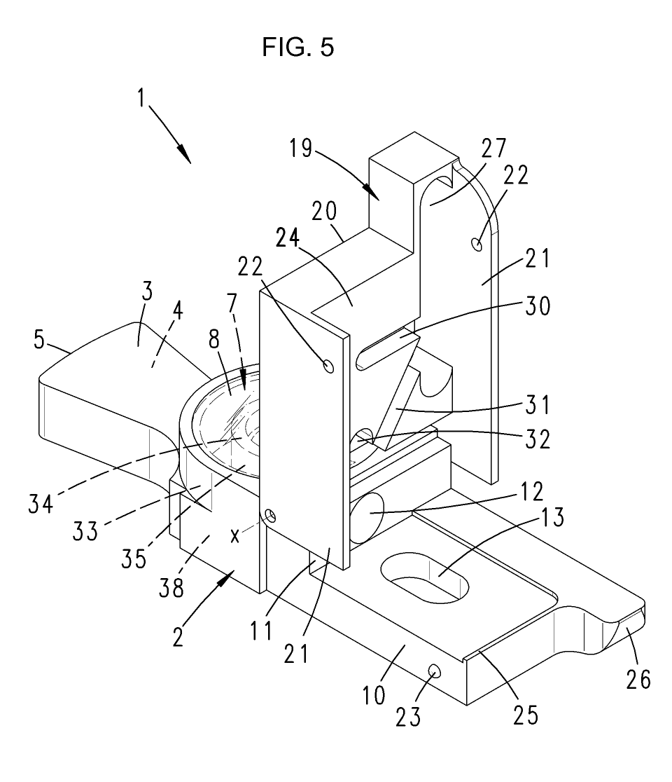

FIG. 5 shows a perspective illustration of the dispenser in a standby position for being fitted with a container part;

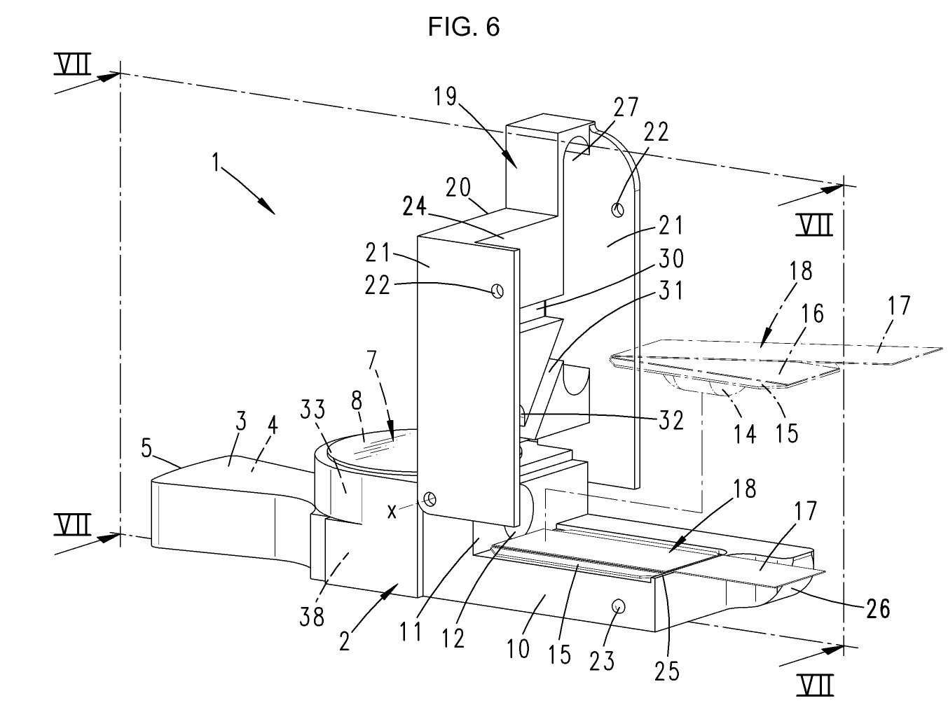

FIG. 6 shows a further perspective illustration of the dispenser in the fitting-standby position;

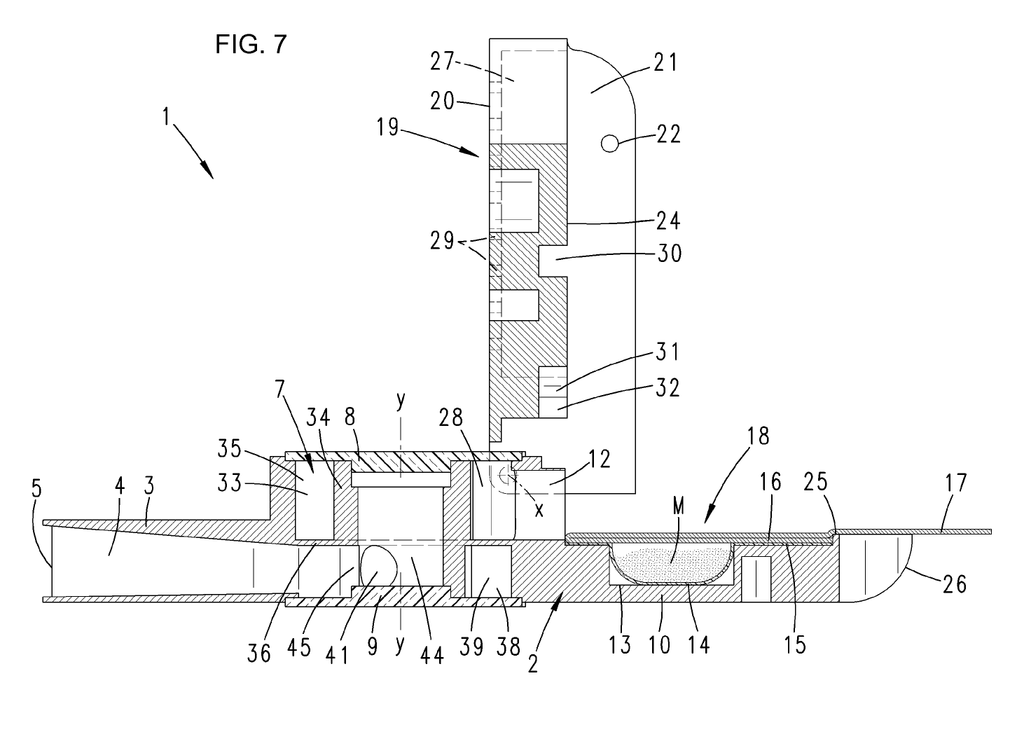

FIG. 7 shows the section along section plane VII-VII in FIG. 6;

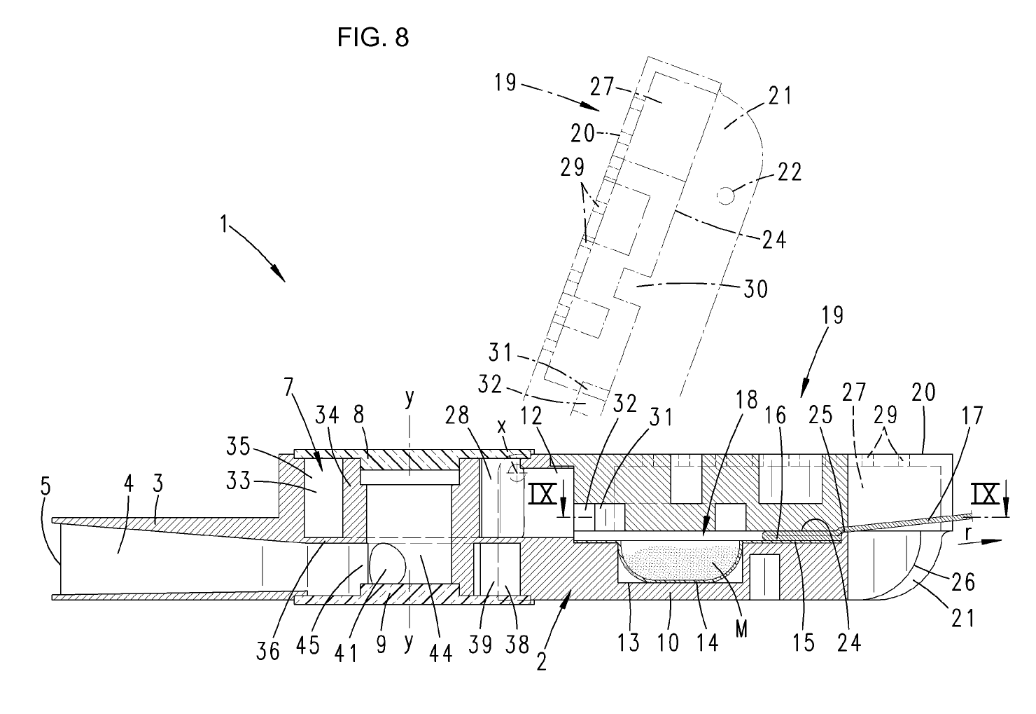

FIG. 8 shows a sectional illustration corresponding to FIG. 7, but relating to the dispenser-standby position;

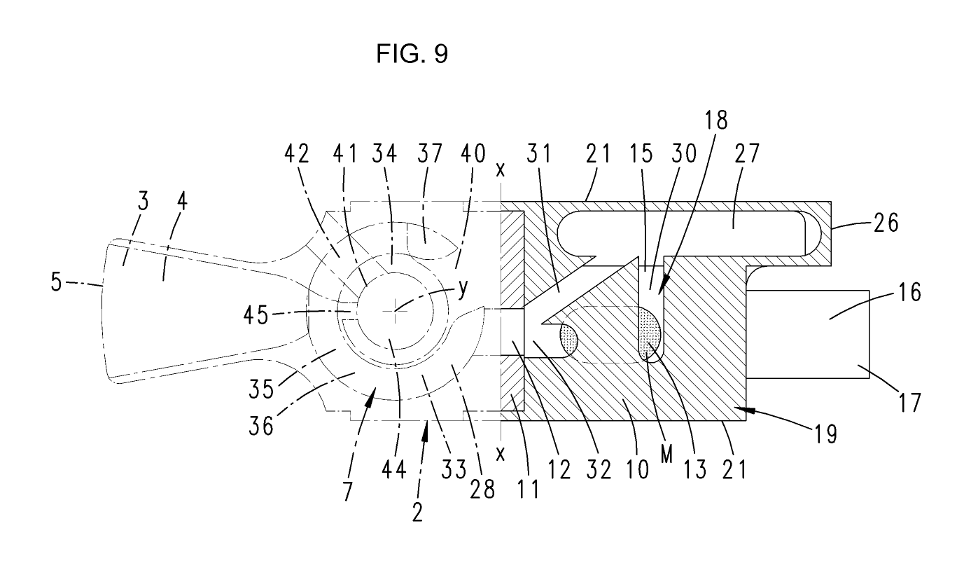

FIG. 9 shows the section along line IX-IX in FIG. 8;

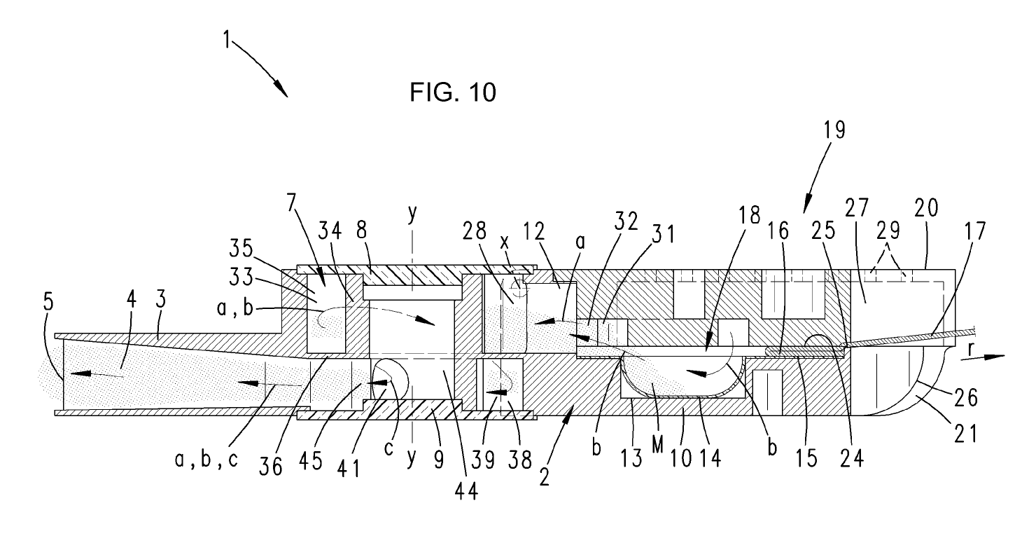

FIG. 10 shows a sectional illustration corresponding to FIG. 8, but during an inhaling process;

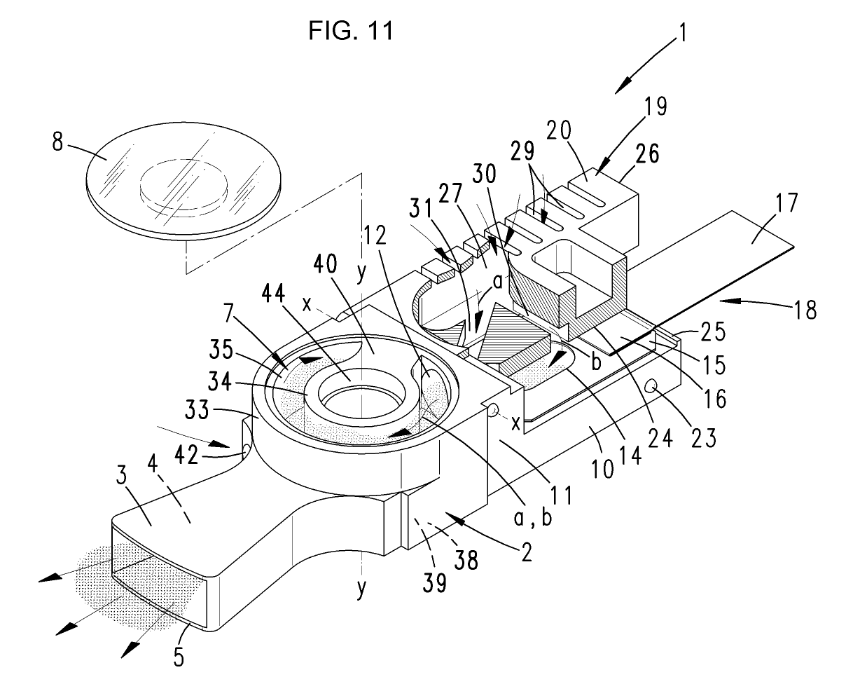

FIG. 11 shows a partially exploded, partially sectional perspective illustration of the dispenser during the inhaling process;

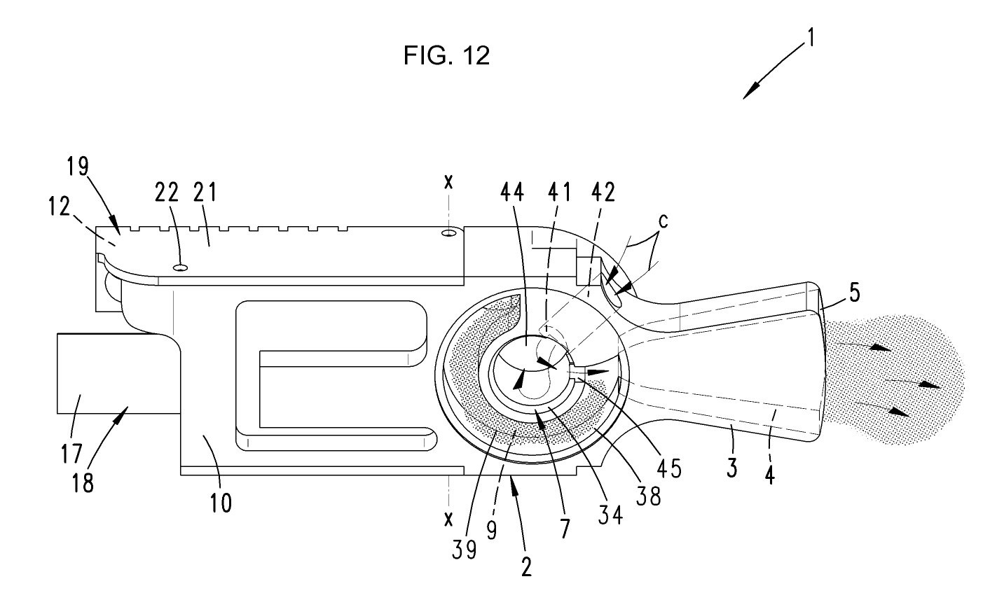

FIG. 12 shows the perspective view from beneath of the dispenser during the inhaling process;

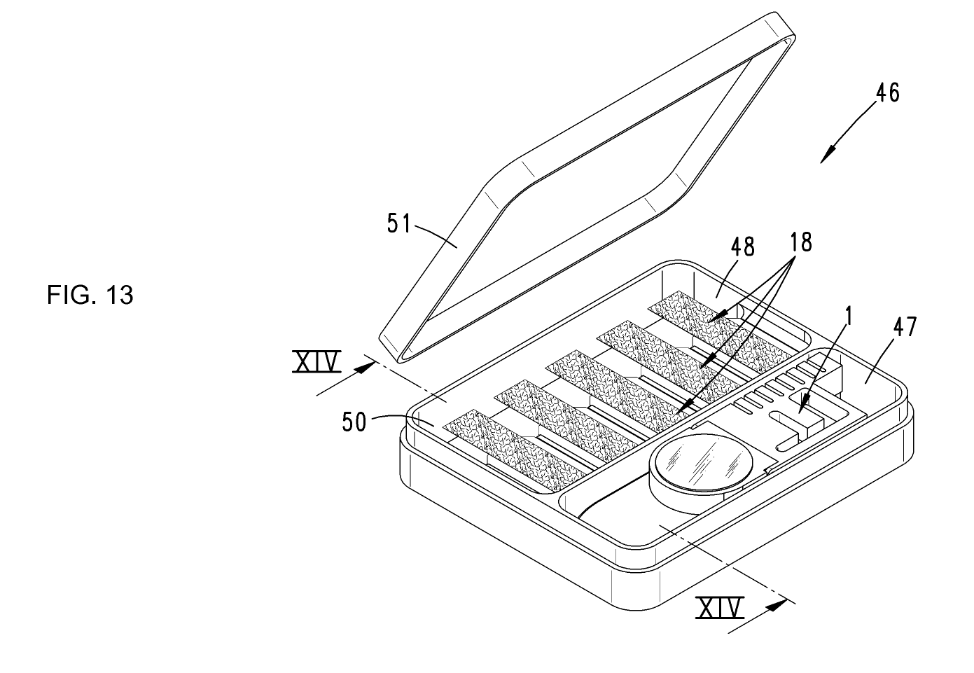

FIG. 13 shows a perspective illustration of a storage box for accommodating the dispenser and a plurality of container parts to be associated with the dispenser; and

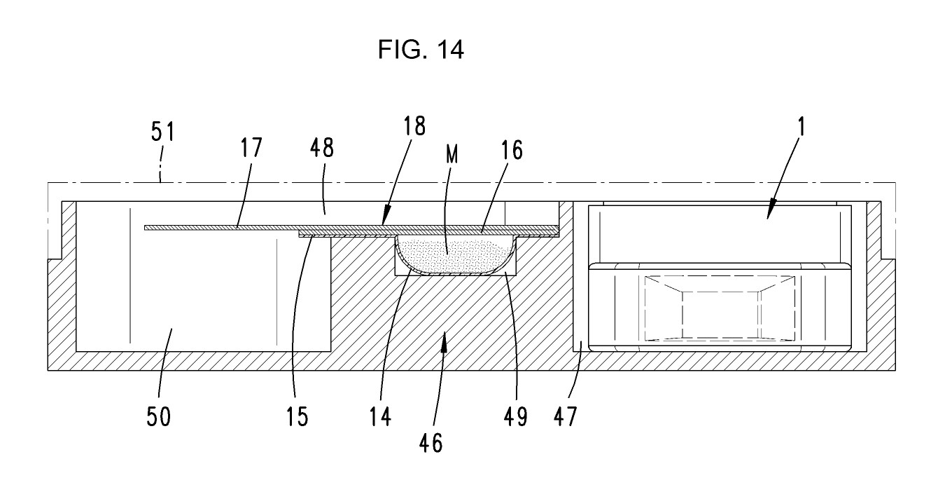

FIG. 14 shows the section along line XIV-XIV in FIG. 13.

A dispenser 1 along the lines of an inhaler, realized as a device which is convenient to carry in one's pocket, will be illustrated and described, in first instance with reference to FIG. 1. This device has a substantially elongate rectangular housing 2 with a length/width ratio of approximately 2:1 to 2.5:1 and a height, as seen perpendicularly to the longitudinal extent, corresponding approximately to a quarter of the longitudinal extent. The parts of the dispenser 1 are realized as plastics injection moldings.

A mouthpiece 3 projects from the housing 2 in continuation of the longitudinal extent of the latter. With a single-piece configuration overall, the transition from the housing 2 to the mouthpiece 3, in relation to the widthwise extent of the housing 2, is waisted.

The mouthpiece 3 has a suction channel 4 passing through along its longitudinal extent, the suction channel 4 terminating in a suction mouth 5 at the outlet end.

When the dispenser 1 is not in use, the mouthpiece 3 can be covered over, in accordance with the illustrations in FIGS. 1 and 2, by a closure cap 6. The latter takes up the width of the housing 2 and can be plugged on in the region of the waist-like transition from the mouthpiece 3 to the housing 2.

A round vortex chamber 7 is formed in the housing 2 in association with the transition region from the housing 2 to the mouthpiece 3. This vortex chamber extends over two levels over the entire height of the housing 2, the chamber cover 8 and the chamber base 9 being formed by circular-disc-shaped covering plates which can be plugged on the housing 2. In the exemplary embodiment illustrated, the chamber cover 8 and chamber base 9 are transparent and are plugged on the housing 2 such that they can be removed for easy cleaning of the vortex chamber 7.

The housing part which is directed away from the mouthpiece 3, and adjoins the vortex chamber 7, is of stepped formation heightwise. The remaining surface of this housing part 10, this surface being offset vertically in relation to the cover 8 of the vortex chamber, extends in a center plane for example in relation to the vertical extent of the housing 2.

At the end, i.e. directed toward the offset plane of the housing part 10, an air channel 12 opens out in the step 11, which adjoins the vortex chamber 7 on the side opposite to the mouthpiece 3, this air channel being oriented along the longitudinal extent of the housing 2. This air channel has a diameter adapted to the height of the step. At its other end, the air channel 12 opens out in the vortex chamber 7.

A holder 13 is formed in the housing part 10 and opens in the direction of the planar surface. It has a slot-like outline in plan view and is also formed, at a distance from the air-channel mouth in step 11, in axial continuation of the air channel 12. The contour and depth of the holder 13 are adapted to the contour and height of a container part 14 which is to be accommodated.

As is evident from the further illustrations, the container part 14 is configured in the manner of a blister pack, the container part 14 storing a pulverulent substance M. For this purpose, the container part 14 is produced in a bowl-like manner from a plastics material, a supporting portion 15 with a planar surface also adjoining the encircling opening periphery of the container part 14. Overall, the container part 14 is formed from the supporting portion 15 as an indentation.

The substance M stored in the container part 14 is sealed-in, in the not-in-use position, by an aluminum foil which covers over the supporting portion 15 and the container part 14 over the entire surface area and constitutes the container covering 16. This container covering 16 can be pulled off in order to uncover the container part 14 and/or the substance M stored therein, for which purpose the aluminum foil or the container covering 16, starting from a narrow region of the supporting portion 15 oriented transversely to the longitudinal extent of the container part 14, continues freely in the opposite direction and covers over that region of the container covering 16 which seals the container part 14 and the supporting portion 15, resting freely on this region of the container covering 16 in the process. The free end of the container covering 16 projects freely beyond that end peripheral edge of the supporting portion 15 which s directed away from the region of inflection of the container covering 16, and it forms a pull-off handgrip 17 in the process.

The holder 13 for the container part 14, this holder being formed in the housing part 10, is positioned in a region which is offset at a lower vertical level than the surface of the housing part. The vertical offset corresponds substantially to the material thickness of the blister 18 outside the container part 14, the blister being made up of the container part 14, container covering 16 and supporting portion 15. Furthermore, in particular the length of the recess, as seen along the longitudinal extent of the housing 2, is adapted to the length of the supporting portion 15, as seen along the longitudinal extent of the container part 14. That surface of the recess which surrounds the opening 13 serves as a support for supporting portion 15 when the blister 18 is placed in position.

An opening flap 19 is associated with the housing part 10. This opening flap is articulated on the housing part 10 in the region of the step 11 such that it can be swung about an axis x directed transversely to the longitudinal extent of the housing. The opening flap 19 has a flap cover 20. Side walls 21 running on both sides along the longitudinal extent of the housing 2 are formed in one piece with the flap cover 20 and, in the closed position of the flap, flank the associated side surfaces of the fixed housing part 10.

The opening flap 19 is latched in the flap-closure position, for which purpose latching recesses 22 are provided in the side walls 21, and protrusions 23 with lens-shaped heads on the housing part penetrate into these recesses. The resulting latching action can easily be disabled by the user for the purpose of opening the flap 19.

In the flap-closure position, the underside 24 of the cover rests with surface contact on the facing upper side of the housing part 10, that is to say at least on the planar surface surrounding the recess which accommodates the holder 13. With the blister placed in position in the recess in the housing part and, at the same time, the vertical offset in the housing part 10 being balanced out, the underside 24 of the cover is correspondingly located on the blister 18, in particular on the container covering 16, which forms the pull-off handgrip 17 in the free end region. Accordingly, the blister 18, once placed in position, is secured in the flap-closure position on the one hand by virtue of the container part 14 being accommodated in a more or less positively locking manner in the holder 13 and also, on the upper side and underside, by the flap cover and the associated surface of the recess in the housing part, furthermore lateral support of the blister 18 via the supporting portion 15 being achieved, on the one hand, by the step 11 and the associated side wall 21 of the opening flap 19 and, on the other hand, by the peripheral boundary of the recess.

The blister 18 should be positioned in the dispenser 1 such that the folded-over edge of the container covering 16, being aligned parallel to the flap axis x, is directed toward the air channel 12 and, furthermore, the freely extending pull-off handgrip 17 projects in the opposite direction, beyond the boundary of the recess, out of the housing 2, in doing so passing through a slot-like pull-off opening 25 which is left correspondingly between the housing part 10 and the opening flap 19 when it is pivoted into the closure position. This pull-off opening 25 has its width, as seen transversely to the longitudinal extent of the housing 2, adapted to the width of the container covering 16. The vertical height of the pull-off opening 25 corresponds substantially to the material thickness of the container covering 16, so that the latter closes the pull-off opening 25.

Correct orientation of the blister 18 to be placed in position in the dispenser 1 is achieved by the holder 13 being disposed eccentrically along the longitudinal extent of the dispenser 1 and/or of the recess in the housing part 10. Accordingly; the supporting portion 15 of the blister 18 has different leg lengths adapted along the longitudinal extent to the eccentric arrangement of the holder 13, so that the blister 18 can be placed in position exclusively in the predetermined orientation.

The pull-off opening 25 is formed in a region of the housing part 10, and of the opening flap 19, which is set back in the direction of the holder 13 in relation to the rear wall 26 of the device, so that in the standby position, i.e. in the flap-closure position with the blister 18 placed in position, the pull-off handgrip 17, which is exposed in the outward direction, through the pull-off opening 25, in a tab-like manner, is flanked on one side by the housing-part/opening-flap portion having the rear wall 26.

That region of the opening flap 19 which carries the rear wall 26 and projects beyond the portion forming the pull-off opening 25 is provided with a collecting channel 27 which runs along the longitudinal extent of the housing 2 and of the 25, opening flap 19. This collecting channel 27 opens in first instance over its entire surface area towards the underside 24 of the cover and is bounded on a longitudinal side by the associated side wall 21. On the upper side, the collecting channel 27 is substantially covered over by the flap cover 20. For connecting the collecting channel 27 to the surroundings, the flap cover 20 has groove-like inlet openings 29 passing through it, these openings being aligned parallel to the flap axis x and opening, correspondingly, in the upward direction and also laterally, passing through the associated side wall 21 in the process. This gives rise to an inlet-grid-like covering of the collecting channel 27, and in the exemplary embodiment illustrated eight groove-like inlet openings 29 are distributed uniformly over the length of the collecting channel 27.

A branch-like container channel 30 extends transversely to the collecting channel 27. This container channel 30 is configured to be open, in first instance, in the direction of the underside 24 of the cover, in the same way as the collecting channel 27. Once the opening flap 19 has been pivoted into the closure position, both the collecting channel 27 and the container channel 30 are closed-off on the base side by the facing surface of the housing part 10 and, in the case of the container channel 30, by the facing surface of the blister 18.

In respect of the flap-closure position, the container channel 30 extends as far as a position in which it overlies the container part 14, this being associated, furthermore, with that end region of the placed-in-position container part 14 which is directed away from the step 11.

Furthermore, a direct channel 31 which is likewise formed in the opening flap 19, and is likewise open towards the underside 24 of the cover when the flap is in the open position, extends from the region in which the container channel 30 is rooted in the collecting channel 27. As seen in the plan view according to the illustration in FIG. 4, this direct channel 31 extends, enclosing approximately an angle of 30° in relation to the longitudinal extent of the collecting channel 27, in the direction of a combining portion 32 which, in the closed position of the flap, is associated with the air channel 12 in the step of the housing part 10. This combining portion 32 and the direct channel 31 are closed at the base, in the flap-closure position, by the associated blister surface (container covering 16 and supporting portion 15).

In the flap-closure position, the combining portion 32, which is formed in the flap and opens in the direction of the underside 24 of the cover, extends from the air channel 12, which opens out at the step, into a position in which it coincides with that end portion of the holder 13, or of the container part 14 accommodated in the holder 13, which is directed toward the step 11.

The air channel 12, which is formed in the housing and opens in the direction of the housing part 10, opens out, at the other end, in a deflecting portion 28 of the vortex chamber 7, and further in the upper level 33 of the vortex chamber 7, this upper level 33 extending substantially above the separating plane between the housing part 10 and opening flap 19.

The upper level 33 of the vortex chamber 7 is substantially annular, for which purpose an annular wall 34 is provided centrally to extend over the entire height of the upper level 33. Starting from the air-channel entrance, the annular channel 35 of the upper level 33 extends approximately over an angle of 270° (in the clockwise direction in relation to the illustration in FIG. 4) before the annular space 35, after passing through an aperture 37 which opens the base 36 of the level in question, merges into an annular space 39, which is similarly formed in the level 38 disposed beneath. This annular space 39 is also bounded radially on the inside by the annular wall 34, which runs through substantially over the entire vertical height of the vortex chamber 7.

The opening 37 in the base is followed immediately downstream, in the region of the upper level 33, by a partition wall 40 which closes the annular space in the direction of circumferential flow in relation to the entrance of the air channel 12 and extends radially from the associated annular-wall portion.

From the opening 37 to the upper level 33, the annular space 39 of the lower level 38 likewise extends over approximately 270° as far as the port to the suction channel 4 of the mouthpiece 3.

This gives rise to substantially helical guidance of air in the vortex chamber 7, and this is done with the air which is sucked through the vortex chamber 7 circulating through a total of 540°, this furthermore including passage through two planes disposed one beneath the other.

An outside-air opening 41 opens out into the central region of the vortex chamber 7, this central region being left in the center of the vortex chamber 7 by the annular wall 34 and being screened by the annular wall 34 substantially in relation to the annular spaces 35 and 39, and the outside-air channel 42 of this outside-air opening is oriented radially in relation to the vortex-chamber axis y. The free end of the outside-air channel 42 opens out in the waist-like transition region from the housing 2 to the mouthpiece 3, as a result of which the outside-air channel 42, or an intake direction defined by the latter through the channel 42, is directed, with the inclusion of an angle of approximately 45° in relation to a longitudinal axis of the housing, more or less counter to the main air flow of the dispenser 1, this main air flow being substantially in the same direction as the longitudinal extent of the housing 2 and/or a pull-out direction r of the container covering 16, that is to say, furthermore, starting substantially from the rear wall 26 of the housing 2 or of the opening flap 19 and extending up to the suction mouth 5 at the mouthpiece end.

In order to inhale a substance M, in first instance, once the opening flap 19 has been pivoted open into the open position according to FIG. 7, the blister 18 is placed in position such that the container part 14 is accommodated in the holder 13, with the supporting portion 15 of the blister being supported on the associated, recessed surface of the housing part 10. The freely projecting pull-off handgrip 17 extends freely beyond the end of the housing for operating purposes.

Once the opening flap 19 has been closed, the blister 18 is secured in the housing 2. The container part 14 is then opened by virtue of the container covering 16 being pulled off with unrolling action, for which purpose the pull-off handgrip 17, which projects out through the pull-off opening 25, is pulled in pull-off direction r. This pull-off operation is visible, in the exemplary embodiment illustrated, as a result of the opening flap 19 being transparent. The opening flap 19 has a for example colored marking line 43, up to which the container covering 16 has to be pulled. Accordingly, this shows the user how to use the dispenser 1 properly. Pulling-off of the container covering 16 beyond the desired position is prevented by a non-detachable connection between the container covering 16 and the supporting portion 15 at the end.

Once the container covering 16 has been pulled off from the container part 14, the substance M in the housing 2 is exposed. As a result of the covering being absent, the container part 14, then, is connected in terms of flow, at one end, to the direct channel 31, which leads to the collecting channel 27, and, at the other end, to the combining portion 32, so that the container part 14 then connects the direct channel 31 in terms of flow to the combining portion 32, and thus forms part of the channel.

For inhalation purposes, the dispenser 1 is held in the manner of a whistle, preferably between the thumb and forefinger. By virtue of the user sucking in via the mouthpiece 3, air enters into the collecting channel 27 through the inlet openings 29, whereupon the incoming air divides up for passing through the direct channel 31 and for passing through the container channel 30. The pulverulent substance M stored in the container part 14 is discharged by means of this container channel 30, whereupon the substance-containing fraction of air b in the combining portion 32 combines with the fraction of air a through the direct channel 31. Passage through the helical, double-level vortex chamber 7 gives rise to uniform distribution of the substance M in the air stream. Prior to exiting from the vortex chamber 7 to the suction channel 4 of the mouthpiece 3, further outside air c enters via the outside-air opening 41. The outside air c, which is taken in here in the manner of additional air, passes through the outside-air channel 42 into the central space 44 of the vortex chamber 7 and is sucked in via a throughflow opening 45 which is oriented in the direction of the lower annular space 39, directly toward the suction channel 4, and this results in the outside air being combined with the substance-laden air stream a, b from the housing 2 immediately upstream of the transition into the suction channel 4.

During the inhaling process, the free container-covering tab closes the pull-off opening 25, and this works against additional air being taken in.

As a result of the configuration described, air is taken in and/or guided within the housing 2 while bypassing the container covering 16, which is displaced into the pull-off position for inhalation purposes and, at the same time, closes the pull-off opening 25 during inhalation. The suction-air stream which removes the substance M from the container part 14 is guided without causing any interference.

As is illustrated in FIGS. 13 and 14, the dispenser 1 may be accommodated in a portable storage box 46, for which purpose the latter has an accommodating compartment 47 formed in accordance with the plan-view outline of the dispenser 1. A compartment 48 adjacent to the accommodating compartment 47 stores a multiplicity of blisters 18, in the exemplary embodiment illustrated five blisters 18.

The compartment 48 has holders 49, corresponding to the holder 13 in the housing 2, for holding the blisters 18 securely. The container parts 14 of the blisters 18 are placed in position in these holders 49.

For ease of removal of the blisters 18, the free ends of the blisters project freely over a cavity 50 formed in the compartment 48.

The storage box 46 can be closed by a lid 51.

The storage box 46 is of a manageable size overall, and this allows it to be carried around, for example, in a jacket pocket.

All features disclosed are (in themselves) pertinent to the invention. The disclosure content of the associated/attached priority documents (copy of the prior application) is hereby also included in full in the disclosure of the application, also for the purpose of incorporating features of these documents in claims of the present application.

### LIST OF DESIGNATIONS

1 Dispenser
2 Housing
3 Mouthpiece
4 Suction channel
5 Suction mouth
6 Closure cap
7 Vortex chamber
8 Chamber cover
9 Chamber base
10 Housing part
11 Step
12 Air channel
13 Holder
14 Container part
15 Supporting portion
16 Container covering
17 Pull-off handgrip
18 Blister
19 Opening flap
20 Flap cover
21 Side walls
22 Recess
23 Protrusion
24 Underside of the cover
25 Pull-off opening
26 Rear wall
27 Collecting channel
28 Deflecting portion
29 Inlet opening
30 Container channel
31 Direct channel
32 Combining portion
33 Upper level
34 Annular wall
35 Annular space
36 Base of the level
37 Opening
38 Lower level
39 Annular space
40 Partition wall
41 Outside-air opening
42 Outside-air channel
43 Marking line
44 Central space
45 Throughflow opening
46 Storage box
47 Accommodating compartment
48 Compartment
49 Holder
50 Cavity
51 Cover
a Air stream
b Air stream
c Air stream
M Substance
r Pull-off direction
x Flap axis
y Vortex-chamber axis

1-16. (canceled)

17. A dispenser for dispensing pulverulent substances, the assembly comprising:
a dispenser formed with a chamber for accommodating a package having a container part containing a pulverulent substance to be dispensed and a pull-off container covering disposed to be pulled off from the container part in a given pull-off direction, wherein the pulverulent substance may be suctioned out of the container part by an aspirated air stream being aspirated, at least in part, through the container part following removal of the container covering from the container part,
said dispenser having a collecting channel for incoming air, a suction mouth, and having a pull-off opening, wherein the container covering projects out of said pull-off opening of said dispenser for actuating purposes;
said dispenser being formed with a direct channel leading from said collecting channel to said suction mouth and with a container channel leading to said container part, wherein incoming air enters into said collecting channel via a plurality of openings and, from there, splits up into said direct channel and said container channel to cause an air stream to flow laterally in a direction transverse to the pull-off direction, and a combining portion leading from said container part and merging into an end of said direct channel.

18. The dispenser according to claim 17, wherein said dispenser is formed with a vortex chamber, said container channel and said direct channel open into said vortex chamber and merge upstream of said vortex chamber in a direction air flow.

19. The dispenser according to claim 17, wherein said dispenser is formed with a vortex chamber having an upper level and a lower level, and wherein one or both of said container channel or said direct channel enters into said upper level of said vortex chamber.

20. The dispenser according to claim 17, which comprises an opening flap formed with shaped structures which, in conjunction with said accommodating chamber for said container part, create the desired air paths.

21. The dispenser according to claim 20, wherein said opening flap is transparent.

22. The dispenser according to claim 20, wherein said dispenser is formed with inlet openings that are, in part, upward open and, in part, laterally open.

23. The dispenser according to claim 17, wherein the air stream, following passage through said container part, enters, in the same plane, into a deflecting portion of a vortex chamber, the vortex chamber extending over two levels arid, furthermore, one or more additional outside-air openings opening into the vortex chamber.

24. The dispenser according to claim 17, wherein said package is a blister pack.
